(19) United States
(12) Patent Application Publication
(10) Pub. No.: US 2021/0380638 A1
(43) Pub. Date: Dec. 9, 2021

(54) A method for improving aggregate removal by Protein A chromatography

(71) Applicant: WUXI BIOLOGICS IRELAND LIMITED

(72) Inventors: Yifeng LI, Shanghai (CN); Ying WANG, Shanghai (CN); Yuan ZHANG, Shanghai (CN); Weichang ZHOU, Shanghai (CN)

(21) Appl. No.: 17/416,391

(22) PCT Filed: Dec. 20, 2019
(86) PCT No.: PCT/CN2019/127022

§ 371 Date: Jun. 18, 2021

(30) Foreign Application Priority Data
Dec. 21, 2018 (CN) PCT/CN2018/122748

(51) Int. Cl. C07K 1/22; C07K 16/00; B01D 15/38; B01D 15/42

(52) U.S. Cl. CPC C07K 1/22; B01D 15/426; B01D 15/3809; C07K 16/00

(57) ABSTRACT

Protein A chromatography is generally less effective in removing antibody aggregates under typical conditions. Provided is a combination and a method that can significantly improve Protein A's aggregate removal capability. The combination comprises polyethylene glycol (PEG) and a salt (chaotropic or kosmotropic) as wash and elution buffer additives. The synergistic effect of salt and PEG results in almost complete separation of monomer from aggregates. For the case used for demonstration, in comparison with the control run the optimized procedure reduces aggregates in elution pool from 20% to 3-4%. This new method, by facilitating aggregate removal at the capture step, improves the overall robustness of downstream process.

The present application is a U.S. National Stage entry of PCT Application No. PCT/CN2019/127022, filed on Dec. 20, 2019, which claims priority to Application No. PCT/CN2018/122748, filed on Dec. 21, 2018, all of which are incorporated in reference herein.

### TECHNICAL FIELD

The present invention relates generally to a combination and a method of removing antibody aggregate by Protein A chromatography.

### BACKGROUND OF THE INVENTION

In general, Protein A chromatography under typical conditions is less effective at removing aggregates. Although aggregates are known to bind more strongly than monomer (D. Yu, Y. Song, R. Y. Huang, et al., Molecular perspective of antibody aggregates and their adsorption on Protein A resin, J. Chromatogr. A, 2016, 1457, 66-75), they are often co-eluted with the latter and adjusting elution pH alone usually is not sufficient for good separation. Consequently, in many cases aggregate removal relies on a single polishing chromatography post Protein A. However, relying on a single step for aggregate removal is unfavorable as such design decreases the robustness of the entire downstream process.

In certain case the yield of the step dedicated to aggregate removal needs to be greatly sacrificed in order to meet the purity requirement. This relying on single-step design is especially problematic for projects with higher-than-average aggregate content. It is desirable to have a complementary aggregate-removing step that can share the burden and partially clear the aggregates at an early stage.

### DISCLOSURE OF THE INVENTION

The present invention provides a combination for use in Protein A chromatography comprising a component A which is at least one type of polyethylene glycol (PEG) polymer and a component B which is at least one Hofmeister series salt (e.g. chaotropic salts or kosmotropic salts).

In one embodiment, the combination consists of a component A which comprises or preferably is at least one type of polyethylene glycol (PEG) polymer and a component B which comprises or preferably is at least one Hofmeister series salt.

In one embodiment, ratio of the PEG and the salt ranges from 1 g:2.5 mmol to 1 g:100 mmol, preferably from 1 g:10 mmol to 1 g:25 mmol.

In one embodiment, the component of combination such as component A or component B may be formulated separately. In one embodiment, the component of combination such as component A or component B may be formulated as a homogenous composition.

In one embodiment, the molecular weight of PEG polymer ranges from about 200 Dalton to about 10,000,000 Dalton, preferably from about 400 Dalton to about 6000 Dalton. For example, PEG 200 Dalton, PEG 400 Dalton, PEG 600 Dalton, PEG 800 Dalton, PEG 1000 Dalton, PEG 1500 Dalton, PEG 2000 Dalton, PEG 3000 Dalton, PEG3350 Dalton, PEG 4000 Dalton, PEG 6000 Dalton and PEG 8000 Dalton. The PEG that is able to improve Protein A chromatography's antibody aggregate removal together with a Hofmeister series salt is within the scope of the invention.

In one embodiment, the Hofmeister series salt is composed of a combination of Hofmeister series of cations and inions, preferably is one salt selected from the group consisting of calcium chloride, sodium chloride, magnesium chloride, and potassium chloride.

In one embodiment, Protein A chromatography is used to improve protein sample aggregate removal, wherein the protein sample comprises any type of protein which contains an Fc region recognizable by Protein A. Such protein comprises antibodies and Fc-fusion proteins. The antibody could be a monoclonal antibody, or a polyclonal antibody. The antibody could be monospecific, bispecific or multi-specific. The antibody could be a mouse antibody, a chimeric antibody, a humanized antibody or a human antibody. An Fc-fusion protein is composed of an Fc region of an antibody and a genetically linked active protein.

In other aspect, inventors provide a composition or a kit, wherein the combination or the kit further comprises a component C, the component C is one buffer selected from the group consisting of wash buffer solution and elution buffer solution, wherein the wash buffer solution or elution buffer solution comprises NaAc and/or HAc, for example. It will be understood by those skilled in the art that in the present invention PEG and Hofmeister series salts can be dissolved in any background buffer as long as the buffer can be used for wash or elution.

In one specific embodiment, ratio of weight of PEG polymer relative to the volume of the wash buffer solution or elution buffer solution is from about 10 g:1 L to about 100 g:1 L, preferably from about 20 g:1 L to about 50 g:1 L., that is, percentage of weight of PEG polymer in the volume of the wash buffer solution or elution buffer solution is from about 1 w/v % to about 10 w/v %, preferably from about 2 w/v % to about 5 w/v %, such as 1 w/v %, 2 w/v %, 3 w/v %, 4 w/v %, 5 w/v %, 6 w/v %, 7 w/v %, 8 w/v %, 9 w/v %, 10 w/v %; the effective PEG concentration depends on the molecular weight of the particular PEG being used. For example, the required percentage of weight of PEG3350 in the volume of the wash buffer solution or elution buffer solution is from about 3.5 w/v % to about 5 w/v %. A lower percentage is sufficient for PEG polymer with higher molecular weight (e.g., PEG 6000) whereas a higher percentage is required for PEG with lower molecular weight (e.g., PEG 600).

In one specific embodiment, ratio of molar mass of the Hofmeister series salt relative to the volume of the wash buffer solution or elution buffer solution is about 250 mmol:1 L and more, preferably is from about 250 mmol:1 L to about 1 mol:1 L, more preferably is from about 500 mmol:1 L to 750 mmol:1 L, that is, percentage of molar mass of the Hofmeister series salt such as calcium chloride, or sodium chloride, or magnesium chloride, or potassium chloride in the volume of the wash buffer solution or elution buffer solution is about 250 mM and more, preferably is from about 250 mM to about 1 M, more preferably is from about 500 mM to about 750 mM, such as 200 mM, 300 mM, 400 mM, 500 mM, 600 mM, 700 mM, 800 mM, 900 mM and 1 M.

In another aspect, the invention provides aforementioned combination or composition or kit for use in protein sample purification by Protein A chromatography, wherein the combination improves monomer-aggregate resolution on Protein A chromatography column, allowing effective removal of antibody aggregates.

The invention provides the use of the aforementioned combination for preparation of the wash buffer and/or the elution buffer for the Protein A column. In particular, PEG and Hofmeister series salts are used together as wash and/or elution buffer additives to achieve the resolution enhancing effect.

In one embodiment, the component of combination such as component A or component B may be formulated separately. In one embodiment, the component of combination such as component A or component B may be formulated as a homogenous composition.

In one embodiment, the molecular weight of PEG polymer ranges from about 200 Dalton to about 10,000,000 Dalton, preferably from about 400 Dalton to about 6000 Dalton. For example, PEG 200 Dalton, PEG 400 Dalton, PEG 600 Dalton, PEG 800 Dalton, PEG 1000 Dalton, PEG 1500 Dalton, PEG 2000 Dalton, PEG 3000 Dalton, PEG3350 Dalton, PEG 4000 Dalton, PEG 6000 Dalton and PEG 8000 Dalton. The PEG that is able to improve Protein A chromatography's protein sample such as Fc-region containing antibody aggregate removal together with a Hofmeister series salt is within the scope of the invention.

In one embodiment, the Hofmeister series salt is composed of a combination of Hofmeister series of cations and inions, preferably is one salt selected from the group consisting of calcium chloride, sodium chloride, magnesium chloride, and potassium chloride.

In one embodiment, the protein sample comprises any type of protein which contains an Fc region recognizable by Protein A. Such protein comprises antibodies and Fc-fusion proteins. The antibody could be a monoclonal antibody, or a polyclonal antibody. The antibody could be monospecific, bispecific or multi-specific. The antibody could be a mouse antibody, a chimeric antibody, a humanized antibody or a human antibody. An Fc-fusion protein is composed of an Fc region of an antibody and a genetically linked active protein.

In one embodiment, the aforementioned combination further comprises a component C, the component C is one buffer selected from the group consisting of wash buffer solution and elution buffer solution, wherein the wash buffer solution or elution buffer solution comprises NaAc and/or HAc, for example. It will be understood by those skilled in the art that in the present invention PEG and Hofmeister series salts can be dissolved in any background buffer as long as the buffer can be used for wash or elution.

In one specific embodiment, ratio of weight of PEG polymer relative to the volume of the wash buffer solution or elution buffer solution is from about 10 g:1 L to about 100 g:1 L, preferably from about 20 g:1 L to about 50 g:1 L, that is, percentage of weight of PEG polymer in the volume of the wash buffer solution or elution buffer solution is from about 1 w/v % to about 10 w/v %, preferably from about 2 w/v % to about 5 w/v %, such as 1 w/v %, 2 w/v %, 3 w/v %, 4 w/v %, 5 w/v %, 6 w/v %, 7 w/v %, 8 w/v %, 9 w/v %, 10 w/v %; the effective PEG concentration depends on the molecular weight of the particular PEG being used. For example, the required percentage of weight of PEG3350 in the volume of the wash buffer solution or elution buffer solution is from about 3.5 w/v % to about 5 w/v %. A lower percentage is sufficient for PEG polymer with higher molecular weight (e.g., PEG 6000) whereas a higher percentage is required for PEG with lower molecular weight (e.g., PEG 600).

In one specific embodiment, ratio of molar mass of the Hofmeister series salt relative to the volume of the wash buffer solution or elution buffer solution is about 250 mmol:1 L and more, preferably is from about 250 mmol:1 L to about 1 mol:1 L, more preferably is from about 500 mmol:1 L to 750 mmol:1 L, that is, percentage of molar mass of the Hofmeister series salt such as calcium chloride, or sodium chloride, or magnesium chloride, or potassium chloride in the volume of the wash buffer solution or elution buffer solution is about 250 mM and more, preferably is from about 250 mM to about 1 M, more preferably is from about 500 mM to about 750 mM, such as 200 mM, 300 mM, 400 mM, 500 mM, 600 mM, 700 mM, 800 mM, 900 mM and 1 M.

In further aspect, the invention provides a method for removing antibody aggregates by Protein A chromatography, comprising the following steps:

1) loading a protein sample onto a Protein A chromatography column,
2) washing the column with a wash buffer, wherein the wash buffer comprises at least one type of PEG polymer and at least one Hofmeister series salt, and
3) eluting the column with an elution buffer, wherein the elution buffer comprises at least one type of PEG polymer and at least one Hofmeister series salt.

In the method, the PEG polymer has a molecular weight of from about 200 Dalton to about 10,000,000 Dalton, preferably from about 400 Dalton to about 6000 Dalton. For example, PEG 200 Dalton, PEG 400 Dalton, PEG 600 Dalton, PEG 800 Dalton, PEG 1000 Dalton, PEG 1500 Dalton, PEG 2000 Dalton, PEG 3000 Dalton, PEG3350 Dalton, PEG 4000 Dalton, PEG 6000 Dalton, and PEG 8000 Dalton. The PEG that is able to improve Protein A chromatography's protein sample such as Fc-region containing antibody aggregate removal together with a Hofmeister series salt is within the scope of the invention.

In one embodiment, the Hofmeister series salt is composed of a combination of Hofmeister series of cations and inions, preferably is one salt selected from the group consisting of calcium chloride, sodium chloride, magnesium chloride, and potassium chloride.

In one embodiment, the protein sample comprises any type of protein which contains an Fc region recognizable by Protein A. Such protein comprises antibodies and Fc-fusion proteins. The antibody could be a monoclonal antibody, or a polyclonal antibody. The antibody could be monospecific, bispecific or multi-specific. The antibody could be a mouse antibody, a chimeric antibody, a humanized antibody or a human antibody. An Fc-fusion protein is composed of an Fc region of an antibody and a genetically linked active protein.

In one embodiment, the aforementioned combination further comprises a component C, the component C is one buffer selected from the group consisting of wash buffer solution and elution buffer solution, wherein the wash buffer solution or elution buffer solution comprises NaAc and/or HAc, for example. It will be understood by those skilled in the art that in the present invention PEG and Hofmeister series salts can be dissolved in any background buffer as long as the buffer can be used for wash or elution.

In one specific embodiment, ratio of weight of PEG polymer relative to the volume of the wash buffer solution or elution buffer solution is from about 10 g:1 L to about 100 g:1 L, preferably from about 20 g:1 L to about 50 g:1 L, that is, percentage of weight of PEG polymer in the volume of the wash buffer solution or elution buffer solution is from about 1 w/v % to about 10 w/v %, preferably from about 2 w/v % to about 5 w/v %, such as 1 w/v %, 2 w/v %, 3 w/v %, 4 w/v %, 5 w/v %, 6 w/v %, 7 w/v %, 8 w/v %, 9 w/v %, 10 w/v %; the effective PEG concentration depends on the molecular weight of the particular PEG being used. For example, the required percentage of weight of PEG3350 in the volume of the wash buffer solution or elution buffer solution is from about 3.5 w/v % to about 5 w/v %. A lower percentage is sufficient for PEG polymer with higher molecular weight (e.g., PEG 6000) whereas a higher percentage is required for PEG with lower molecular weight (e.g., PEG 600).

In one specific embodiment, ratio of molar mass of the Hofmeister series salt relative to the volume of the wash buffer solution or elution buffer solution is about 250 mmol:1 L and more, preferably is from about 250 mmol:1 L to about 1 mol:1 L, more preferably is from about 500 mmol:1 L to 750 mmol:1 L, that is, percentage of molar mass of the Hofmeister series salt such as calcium chloride, or sodium chloride, or magnesium chloride, or potassium chloride in the of volume the wash buffer solution or elution buffer solution is about 250 mM and more, preferably is from about 250 mM to about 1 M, more preferably is from about 500 mM to about 750 mM, such as 200 mM, 300 mM, 400 mM, 500 mM, 600 mM, 700 mM, 800 mM, 900 mM and 1 M.

### THE FEATURES AND ADVANTAGES OF THIS INVENTION

The inventors have generated a combination and a method of removing antibody aggregates by Protein A chromatography. Protein A's antibody aggregate removing capability is improved significantly by using the combination comprising PEG and Hofmeister series salt such as calcium chloride or sodium chloride. This new method, by allowing the majority of aggregates to be removed at the Protein A capture step, significantly alleviates the burden on subsequent polishing steps and hence improves the overall robustness of downstream process.

### BRIEF DESCRIPTION OF THE DRAWINGS

FIG. 1. Overlay of chromatograms from five Protein A runs. Top, whole elution profiles. Bottom, zoom-in view of the elution peaks. The column was eluted with a linear pH gradient. For each run different amounts of PEG were added to wash and elution buffers.

FIG. 2. Overlay of chromatograms from five Protein A runs. Top, whole elution profiles. Bottom, zoom-in view of the elution peaks. The column was eluted with a linear pH gradient. For each run different amounts of calcium chloride were added to wash and elution buffers.

FIG. 3. Overlay of chromatograms from five Protein A runs conducted with load containing another antibody. Top, whole elution profiles. Bottom, zoom-in view of the elution peaks. For these five runs, the load contains an antibody different from the one used in all other runs and in this case the load contains less than 5% aggregates. The column was eluted with a linear pH gradient. For each run different amounts of calcium chloride were added to wash and elution buffers. These experiments were performed to confirm the trend observed in FIG. 2.

FIG. 4. (A) Overlay of chromatograms from three Protein A runs with low resolution and (B) chromatogram from a Protein A run with significantly improved resolution. The column was eluted with a linear pH gradient. For each run different amounts of calcium chloride (0, 150, 250, 500 mM) with 5% PEG were added to wash and elution buffers. The run with 500 mM calcium chloride and 5% PEG showed dramatically improved monomer-aggregate resolution.

FIG. 5. Overlay of chromatograms from three Protein A runs. The column was eluted with a linear pH gradient. For these three runs, the wash and elution buffers contained 5% PEG, 2 M urea/5% PEG and 0.5 M arginine/5% PEG, respectively.

FIG. 6. Protein A chromatogram under (A) linear gradient and (B) stepwise elution. 500 mM sodium chloride and 5% or 3.5% PEG (for linear and stepwise gradient, respectively) were added to wash and elution buffers to facilitate aggregate removal.

FIG. 7. Overlay of chromatograms from (A) three Protein A runs with low resolution and (B) two Protein A runs with improved resolution. The column was eluted with a linear pH gradient. For each run different amounts of sodium chloride (0 mM, 250 mM, 500 mM, 600 mM and 750 mM) were added to wash and elution buffers. The runs with 600 and 750 mM sodium chloride showed improved monomer-aggregate resolution, but only the run with 600 mM sodium chloride gave acceptable product yield. However, the separation of monomer from aggregates is less complete than that with PEG/sodium chloride combination.

FIG. 8. Overlay of Protein A chromatograms from runs with (A) unoptimized and (B) optimized protocols. For the optimized protocol, 750 mM sodium chloride and 5% PEG were added to wash and elution buffers. With sodium chloride and PEG being added to the mobile phase, separation of the antibody monomer from aggregates was improved. The elution pool SEC purity was improved from 91.1% (unoptimized) to 96.6% (optimized).

### DETAILED DESCRIPTION

In order that the present invention may be more readily understood, certain terms are first defined. Additional definitions are set forth throughout the detailed description.

The term “Polyethylene glycol/PEG” as used in this disclosure, refers to an oligomer or polymer of ethylene oxide. PEG is also known as polyethylene oxide (PEO) or polyoxyethylene (POE), depending on its molecular weight. The structure of PEG is commonly expressed as H—(O—CH₂—CH₂)n-OH. PEGs are commercially available over a wide range of molecular weight from 200 g/mol to 10,000,000 g/mol. For example, molecular weight of the PEG used in this invention ranges from about 400 to about 6000.

The term “protein sample” employed in the present invention refers to a protein which contains an Fc region recognizable by Protein A. Such protein comprises antibodies and Fc-fusion proteins. The antibody could be a monoclonal antibody, or a polyclonal antibody. The antibody could be monospecific, bispecific or multi-specific. The antibody could be a mouse antibody, a chimeric antibody, a humanized antibody or a human antibody. The antibody could be a natural antibody or a recombinant antibody. An Fc-fusion protein is composed of an Fc region of an antibody and a genetically linked active protein.

The term “Fc region” employed in the present invention refers to the fragment crystallizable region of an antibody. Fc region is derived from the constant domains of the antibody's heavy chains. The “Fc region” can be recognized and bound by Protein A.

Exemplary antibodies that could be used in the present invention include Adalimumab, Bezlotoxumab, Avelumab, Dupilumab, Durvalumab, Ocrelizumab, Brodalumab, Reslizumab, Olaratumab, Daratumumab, Elotuzumab, Necitumumab, Infliximab, Obiltoxaximab, Atezolizumab, Secukinumab, Mepolizumab, Nivolumab, Alirocumab, Evolocumab, Dinutuximab, Bevacizumab, Pembrolizumab, Ramucirumab, Vedolizumab, Siltuximab, Alemtuzumab, Trastuzumab, Pertuzumab, Infliximab, Obinutuzumab, Brentuximab, Raxibacumab, Belimumab, Ipilimumab, Denosumab, Ofatumumab, Besilesomab, Tocilizumab, Canakinumab, Golimumab, Ustekinumab, Certolizumab, Catumaxomab, Eculizumab, Ranibizumab, Panitumumab, Natalizumab, Catumaxomab, Bevacizumab, Omalizumab, Cetuximab, Efalizumab, Ibritumomab, Fanolesomab, Tositumomab, Alemtuzumab, Trastuzumab, Gemtuzumab, Infliximab, Palivizumab, Necitumumab, Basiliximab, Rituximab, Capromab, Satumomab, Muromonab, etc.

Exemplary Fc-fusion proteins that could be used in the present invention include Etanercept, Alefacept, Abatacept, Rilonacept, Romiplostim, Belatacept, Aflibercept, etc.

The term “chromatography” refers to any kind of technique which separates an analyte of interest (e.g., an Fc region containing protein such as an immunoglobulin) from other molecules present in a mixture. Usually, the analyte of interest is separated from other molecules as a result of differences in rates at which the individual molecules of the mixture migrate through a stationary medium under the influence of a moving phase, or in bind and elute processes.

The term “Protein A” employed in the present invention encompasses Protein A recovered from a native source, Protein A produced synthetically (e.g., by peptide synthesis or by recombinant techniques), and functional variants thereof. Protein A exhibits high affinity for an Fc region. Protein A can be purchased commercially from Repligen, Pharmacia and Fermatech. Protein A is generally immobilized on a solid phase support material. The term “Protein A” also refers to an affinity chromatography resin or column containing chromatographic solid support matrix to which Protein A is covalently attached.

The term “Hofmeister series salt” refers to salt composed of Hofmeister series of cations (e.g., NH₄⁺, K⁺, Na⁺, Li⁺, Mg²⁺, Ca²⁺, guanidinium⁺) and inions (e.g., SO₄²⁻, HPO₄²⁻, acetate⁻, citrate⁻, Cl⁻, NO₃⁻, Br⁻, I⁻, ClO₄⁻, SCN⁻). Various Hofmeister series salts which may be used in the buffers described herein include, but are not limited to, acetate (e.g. sodium acetate), citrate (e.g. sodium citrate), chloride (e.g. sodium chloride), sulphate (e.g. sodium sulphate), or a potassium salt.

A “buffer” is a solution that resists changes in pH by the action of its acid-base conjugate components. Various buffers which can be employed depending, for example, on the desired pH of the buffer are described in “Buffers. A Guide for the Preparation and Use of Buffers in Biological Systems, Gueffroy, D., ed. Calbiochem Corporation, 1975”. In some steps of the methods of the claimed invention, a buffer has a pH in the range from 2.0 to 4.0, or from 2.8 to 3.8. In other steps of the claimed invention, a buffer has a pH in the range of 5.0 to 9.0. In other steps of the claimed invention, a buffer has a pH in the range of 4.0 to 6.5. In yet other steps of the methods of the claimed invention, a buffer has a pH lower than 4.0. Non-limiting examples of buffers that will control the pH in this range include IVIES, MOPS, MOPSO, Tris, HEPES, phosphate, acetate, citrate, succinate, and ammonium buffers, as well as combinations of these.

The term “wash buffer” refers to the buffer used to wash the chromatography column post sample loading and prior to elution.

The term “elution buffer” refers the buffer used to elute the target protein from the solid phase. The conductivity and/or pH of the elution buffer is/are usually such that the target protein is eluted from the chromatography resin.

### Materials

Calcium chloride dihydrate, sodium acetate trihydrate, sodium chloride, sodium hydroxide and Tris (hydroxymethyl) aminomethane were purchased from Merck (Darmstadt, Germany). Arginine hydrochloride and acetic acid were purchased from J. T. Baker (Phillipsburg, N.J., USA). Polyethylene Glycol (PEG) 3350 and urea were purchased from Sigma-Aldrich (St. Louis, Mo., USA). Mab Select SuRe LX and Tricorn 5/200 column (inner diameter: 5 mm, length: 20 mm) were purchased from GE Healthcare (Uppsala, Sweden). The three antibodies used are intact Immunoglobulin G (IgG). The one used to confirm calcium chloride's effect is IgG4 and the other two are IgG1. All three antibodies used were expressed in CHO—K1 cells grown in HyClone ActiPro culture medium supplemented with Cell Boost 7a and 7b (the medium and feeding supplements are from GE Healthcare) as previously described (X Zhang, T Chen, Y. Li, A parallel demonstration of different resins' antibody aggregate removing capability by a case study, Protein Expr. Purif., 2019, 153, 59-69). For the case used for method development and demonstration, the clarified harvest contains greater than 20% aggregates.

### Equipment

An AKTA pure 150 system installed with Unicorn software version 6.3 (GE Healthcare, Uppsala, Sweden) was used for all chromatographic runs. pH and conductivity was measured using SevenExcellence S470 pH/Conductivity meter (Mettler-Toledo, Columbus, Ohio, USA). Protein concentration was measured using a NanoDrop One spectrophotometer (Thermo Fisher Scientific, Waltham, Mass., USA). An Agilent 1260 liquid chromatography instrument (Agilent Technologies, Santa Clara, Calif., USA) was used for SEC-HPLC analysis.

### Methods

Protein a Chromatography

Mab Select SuRe LX (Protein A affinity medium) was packed in a 0.5 cm diameter column with 15 cm bed height. The column volume (CV) is approximately 3 ml. Recipes of critical buffers for each run are listed in Table 1 (A1: equilibration/wash 1 buffer, A2: wash 2 buffer, B: elution buffer). Protein A load is the culture harvest clarified by depth filtration. For all runs, the column was loaded at 25 mg/ml and run in bind-elute mode. The antibody (IgG) with high percentage of aggregate was eluted with linear (0-100% B over 20 CV) or stepwise gradient. For all runs, after sample loading the column was washed with buffer A1 and A2 each for 3 CV prior to elution. For all chromatographic runs, the system was run at a flow rate of 180 cm/hr (residence time: 5 min). All chromatograms were recorded by monitoring UV absorbance at 280 nm. Elution from selected runs was collected in fractions and analyzed by SEC-HPLC for monomer purity.

TABLE 1
Buffer recipes for Protein A chromatographic runs performed in this study.

| Run NO.^{a} | Buffer recipe |
|---|---|
| 1 | A1: 50 mM Tris-HAc, 150 mM NaCl, pH 7.4 |
| | A2: 50 mM NaAc-HAc, pH 5.5 |
| | B: 50 mM HAc, pH 3.1 |
| 2/3/4/5 | A1: 50 mM Tris-HAc, 150 mM NaCl, pH 7.4 |
| | A2: 50 mM NaAc-HAc, 1.5%/3.0%/5.0%/10% PEG, pH 5.5 |
| | B: 50 mM HAc, 1.5%/3.0%/5.0%/10% PEG, pH 3.1 |
| 6/7/8/9^{b} | A1: 50 mM Tris-HAc, 150 mM NaCl, pH 7.4 |
| | A2: 50 mM NaAc-HAc, 250/500/750/1000 mM CaCl₂, pH 5.5 |
| | B: 50 mM HAc, 250/500/750/1000 mM CaCl₂, pH 3.1 |
| 10/11/12 | A1: 50 mM Tris-HAc, 150 mM NaCl, pH 7.4 |
| | A2: 50 mM NaAc-HAc, 150/250/500 mM CaCl₂, 5% PEG, pH |
| | 5.5 |
| | B: 50 mM HAc, 150/250/500 mM CaCl₂, 5% PEG, pH 3.1 |
| 13/14 | A1: 50 mM Tris-HAc, 150 mM NaCl, pH 7.4 |
| | A2: 50 mM NaAc-HAc, 2M urea/500 mM Arg, 5% PEG, pH |
| | 5.5 |
| | B: 50 mM HAc, 2M urea/500 mM Arg, 5% PEG, pH 3.1 |
| 15 | A1: 50 mM Tris-HAc, 150 mM NaCl, pH 7.4 |
| | A2: 50 mM NaAc-HAc, 500 mM NaCl, 5% PEG, pH 5.5 |
| | B: 50 mM HAc, 500 mM NaCl, 5% PEG, pH 3.1 |
| 16^{c} | A1: 50 mM Tris-HAc, 150 mM NaCl, pH 7.4 |
| | A2: 50 mM NaAc-HAc, 500 mM NaCl, 3.5% PEG, pH 5.5 |
| | B: 50 mM HAc, 500 mM NaCl, 3.5% PEG, pH 3.5 |
| 17/18/19/20 | A1: 50 mM Tris-HAc, 150 mM NaCl, pH 7.4 |
| | A2: 50 mM NaAc-HAc, 250/500/600/750 mM NaCl, pH 5.5 |
| | B: 50 mM HAc, 250/500/600/750 mM NaCl, pH 3.1 |
| 21 | A1: 50 mM Tris-HAc, 150 mM NaCl, pH 7.4 |
| | A2: 50 mM NaAc-HAc, 750 mM NaCl, 5% PEG, pH 5.5 |
| | B: 50 mM HAc, 750 mM NaCl, 5% PEG, pH 3.1 |

Note.
The column was stripped and sanitized with 1M HAc and 0.1M NaOH, respectively.
^{a}The numbers are solely used to distinguish different runs and the actual experiments were not necessarily performed in this order.
^{b}This series of experiments was also conducted with another antibody to confirm the observed trend.
^{c}Stepwise elution.

### Size-Exclusion Chromatography-High Performance Liquid Chromatography (Sec-HPLC)

All samples (Protein A elution fractions and elution pool) were analysed using a Tosoh TSKgel G3000SWx1 stainless steel column (7.8×300 mm). 100 μg of sample was injected per run. The mobile phase consisted of 50 mM sodium phosphate, 300 mM sodium chloride at pH 6.8. Each sample was eluted isocratically for 20 min at a flow rate of 1.0 ml/min. Protein elution was monitored by UV absorbance at 280 nm. The peaks corresponding to the monomer and aggregates were integrated to calculate the percentage of each species.

### Examples

### Example 1: Impact of PEG on Protein a Elution Profile

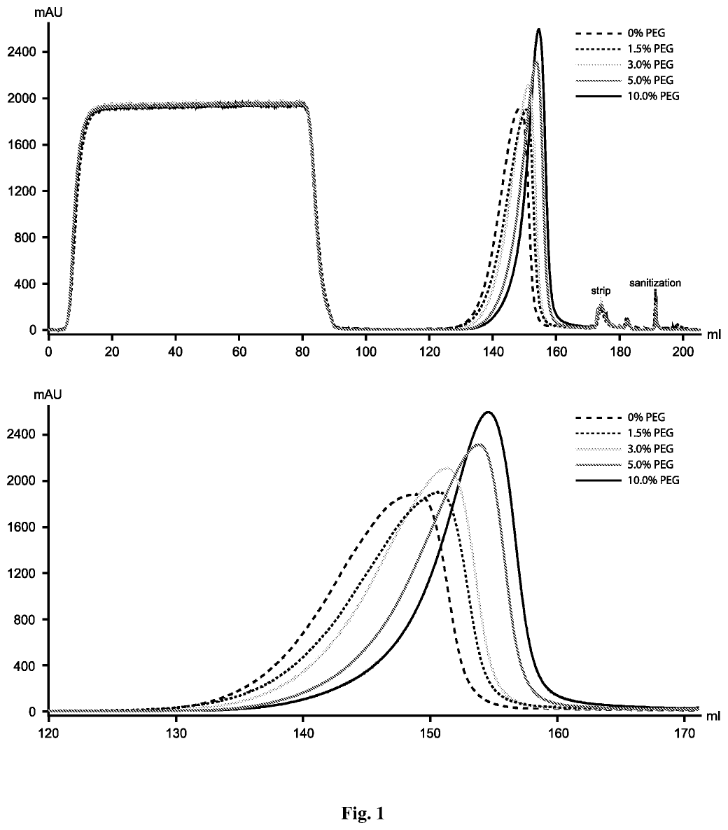

In this study, we first investigated PEG's effect on Protein A elution profile by adding different amounts of PEG (i.e., 1.5%, 3%, 5% and 10%) to wash and elution buffers. With increasing PEG concentration, retention of the aggregation-prone antibody was slightly increased and the elution peak became sharper (FIG. 1). However, different from what is observed on other types of columns (e.g., ion exchange, hydrophobic interaction and mixed-mode) PEG (up to 10%) showed no effect on monomer-aggregate resolution on the Protein A column. This observation explains the lack of pervious report on the application of PEG in Protein A chromatography for aggregate removal.

### Example 2: Impact of Calcium Chloride on Protein a Elution Profile

The inventors designed experiments to explore the effect of calcium chloride on monomer-aggregate resolution as a mobile phase additive. For the case under study, different amounts of calcium chloride (i.e., 250 mM, 500 mM, 750 mM and 1 M) were added to Protein A wash and elution buffers.

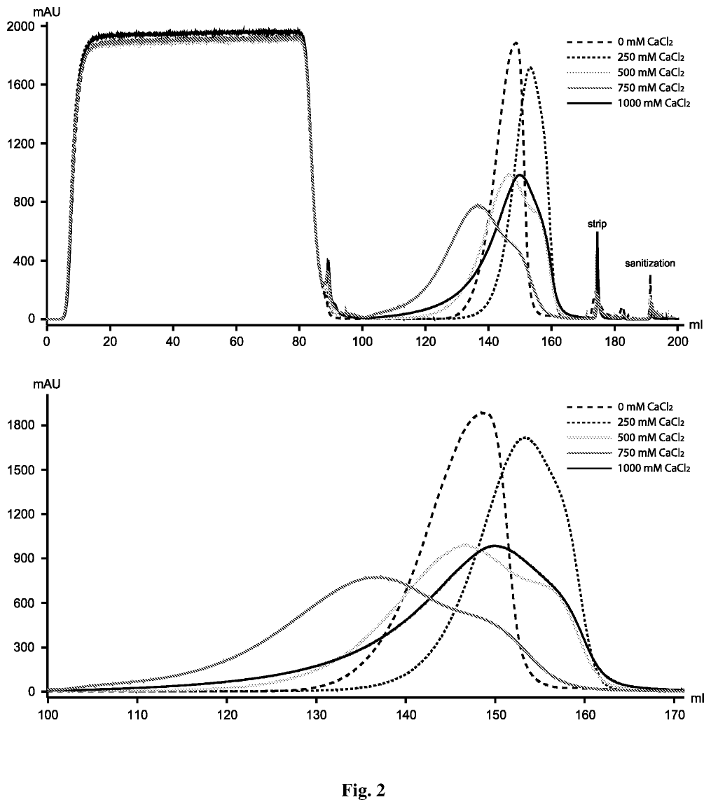

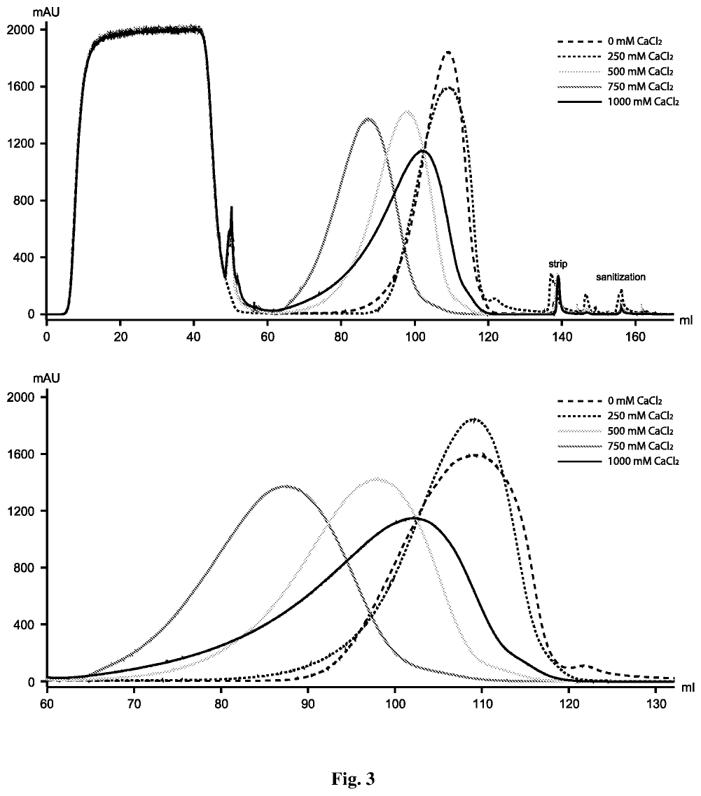

Adding calcium chloride to the mobile phase showed appreciable but not significant impact on both resolution and retention time (FIG. 2). At low concentration (i.e., 250 mM), calcium chloride had little effect on resolution and the elution peak was similar to that of the control run without the salt (in both cases, the elution peak is relatively sharp). Nevertheless, calcium chloride slightly increased the target protein retention time at this concentration. At increased concentrations (i.e., 500 mM and 750 mM), calcium chloride showed a small effect on resolution. Under these two conditions, the elution peak became broader and contained an obvious shoulder. Furthermore, in consistent with previous observation the target protein started to elute at higher pH (750 mM calcium chloride shortened the retention time to a larger degree than 500 mM calcium chloride did). At further increased calcium chloride concentration (i.e., 1 M), the elution peak was as broad as that seen at the two moderate concentrations but the shoulder peak disappeared. More interestingly, the protein retention time was not further shortened and was instead about the same as that at 500 mM calcium chloride. Thus, 750 mM rather than 1 M calcium chloride caused the biggest change to the elution profile in terms of resolution and retention as compared to the control run. The trend at 1 M calcium chloride was somewhat unexpected. To have this result confirmed, the inventors conducted the same experiments (five runs with different amounts of calcium chloride being added to the wash 2 and elution buffers) using another antibody with much lower aggregate content (i.e., <5%). Similar trend was observed: 750 mM rather than 1 M calcium chloride showed the biggest impact on the elution profile (FIG. 3).

It is interesting that calcium chloride improves resolution only at medium concentrations (i.e., 500 mM and 750 mM). It shows no effect on resolution at lower or higher concentrations (i.e., 250 mM and 1 M, respectively). It seems that at low concentration calcium chloride exhibits weak kosmotropic effect and therefore slightly increases retention time. At increased concentrations (i.e., 500 mM and 750 mM), calcium chloride exhibits chaotropic effect and reduces retention time. At these two concentrations, calcium chloride improves monomer-aggregate resolution. At 1 M calcium chloride concentration, the resolution observed at 500 mM and 750 mM diminished and the protein retention time was not further reduced. This suggest that calcium chloride at this high concentration may cause some changes to the target antibody and/or the Protein A ligand, which prevent the interaction between antibody and Protein A from being further weakened.

### Example 3: Synergistic Effect of PEG and Calcium Chloride on Protein a Resolution

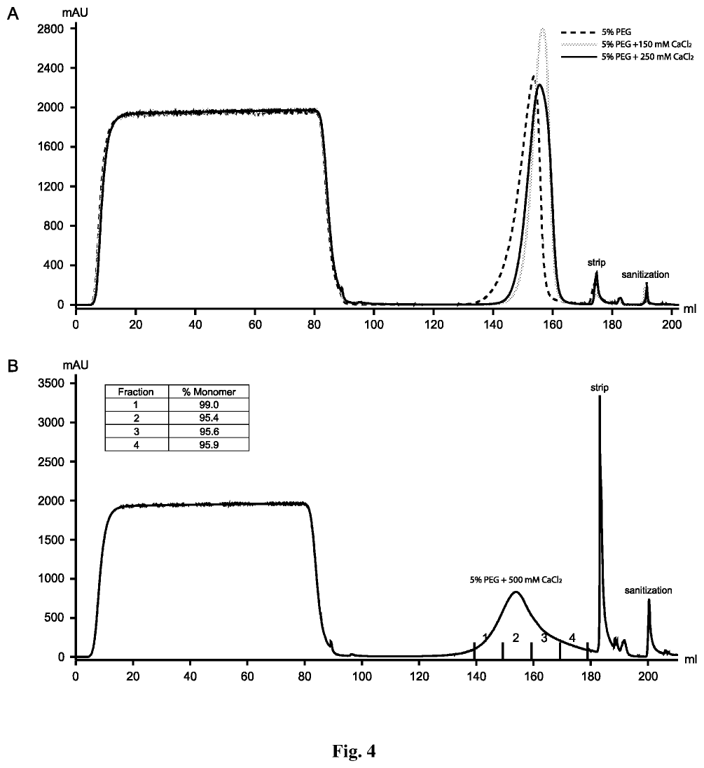

Although calcium chloride at 500 mM and 750 mM improves monomer-aggregate resolution, separation of the two species is far from complete under these conditions. Thus, the inventors next tried PEG/calcium chloride combination. Since PEG itself had little effect on the elution profile at different concentrations, in this study the inventors arbitrarily chose 5% PEG to combine with different amounts of calcium chloride. At low calcium chloride concentrations (i.e., 150 mM and 250 mM), this combination showed no obvious effect and the elution profile is almost identical to that of the run with 5% PEG only (FIG. 4A). However, the combination of 500 mM calcium chloride and 5% PEG showed a remarkable synergistic effect, resulting in significantly improved separation of monomer from aggregates (FIG. 4B). The monomer in the eluate was improved from 80% (control run whose wash 2 and elution buffers contained neither PEG nor calcium chloride) to >96%. The overall protein and monomer yields for this run are 69.5% and 85%, respectively.

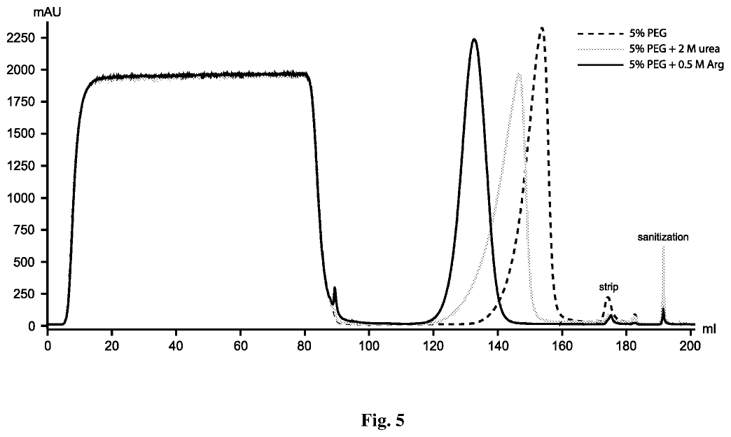

The data suggest that PEG starts to show an enhancing effect when calcium chloride reaches a concentration that improves resolution. Whereas calcium chloride at this concentration can weaken antibody binding to Protein A ligand, its role cannot be replaced by other interaction-weakening agents such as urea or arginine (FIG. 5). Urea and arginine reduce retention time but show no effect on resolution. It seems that calcium chloride's resolution-enhancing capability at medium concentrations is the prerequisite for the observed synergistic effect and PEG's role is to amplify calcium chloride's effect. PEG/magnesium chloride combination can likely achieve the same degree of separation since magnesium ion is close to calcium ion in the Hofmeister series and magnesium chloride showed similar resolution-enhancing effect in the previous study (A. D. Tustian, C. Endicott, B. Adams, J. Mattila, H. Bak, Development of purification processes for fully human bispecific antibodies based upon modification of protein A binding avidity, mAbs 8, 2016, 828-838).

### Example 4: Effect of PEG/Sodium Chloride Combination and Sodium Chloride Alone on Protein a Elution Profile

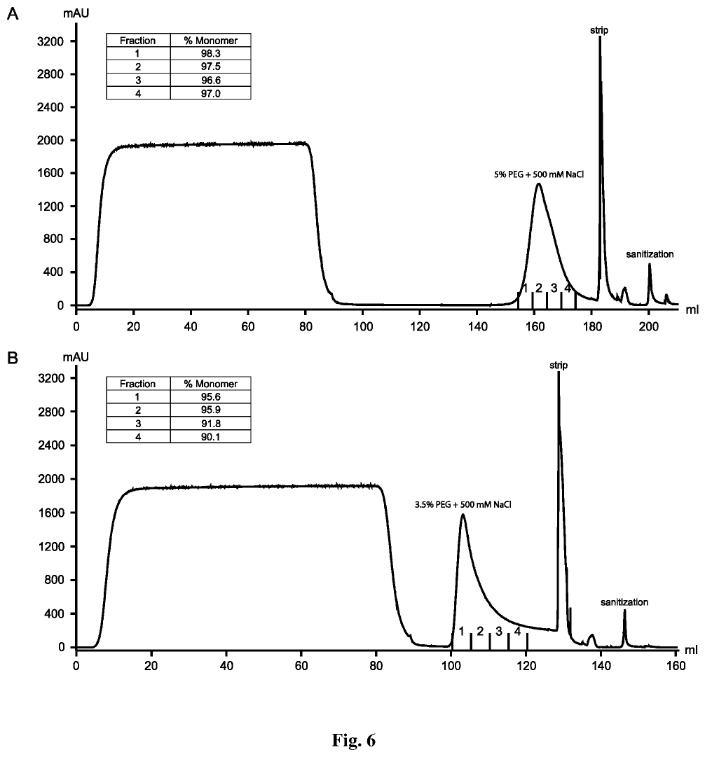

After observing the synergistic effect of PEG and calcium chloride, the inventors also studied the effect of PEG/sodium chloride combination, and received a similar result (FIG. 6A). It can be seen from the figure that the elution peak became sharper in comparison with that from the run with PEG/calcium chloride combination. According to the SEC-HPLC results, PEG/sodium chloride combination also provides slightly better separation. In addition to linear gradient elution, the inventors also developed stepwise elution to facilitate production at large scale (FIG. 6B). The monomer yields for runs with linear and stepwise gradient elution are approximately 88% and 82%, respectively. For the stepwise elution, there is still room for improvement in terms of yield and purity.

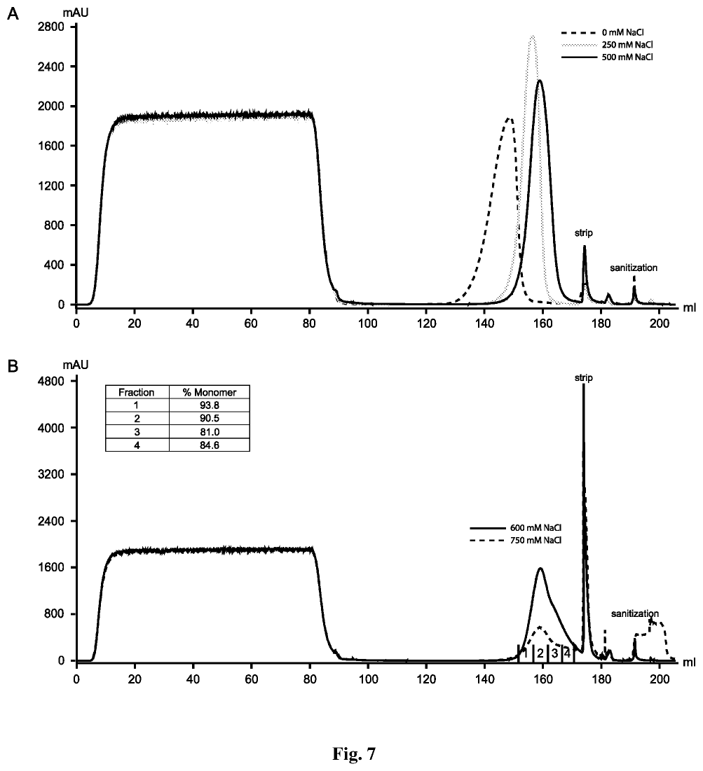

The inventors had learned that PEG alone had no major effect on resolution (FIG. 1). To better understand the effect of PEG/sodium chloride combination, they also studied the effect of sodium chloride alone at different concentrations (i.e., 250 mM, 500 mM, 600 mM and 750 mM). As shown in FIG. 7A, at the two lower concentrations (i.e., 250 mM and 500 mM) sodium chloride increased the protein retention time and the degree of this effect is proportional to the salt concentration. Under these conditions, sodium chloride showed no effect on resolution. When the salt concentration was slightly further increased (i.e., 600 mM), sodium chloride had a dramatic impact on the elution profile and greatly improved the resolution between monomer and aggregates (FIG. 7B, solid line). However, the SEC purity of each fraction is much lower than the corresponding value of fractions from the run with PEG/sodium chloride combination. At further increased sodium chloride concentration (i.e., 750 mM), the product yield significantly dropped (FIG. 7B, dash line). In both cases (i.e., 600 mM and 750 mM sodium chloride), the elution peaks contained a shoulder, suggesting separation of monomer from aggregates is not complete. The existence of a shoulder peak in the elution profiles at both sodium chloride concentrations suggest that better separation is unlikely to be achieved through fine-tuning of sodium chloride concentration. The data indicate that like calcium chloride sodium chloride as a Protein A mobile phase additive can improve monomer-aggregate resolution when reaches certain concentration and the effect can be further improved in the presence of PEG.

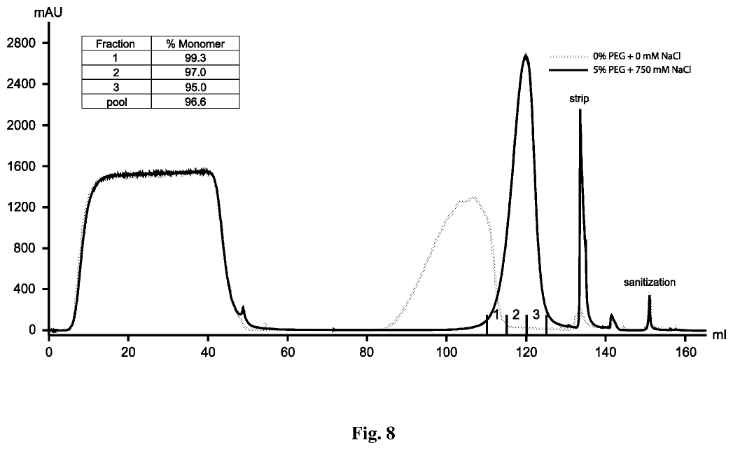

We further confirmed the effect of PEG/sodium chloride combination on resolution enhancement with another case. In this case, the load contains approximately 10% of aggregates. As shown in FIG. 8, the optimized procedure with NaCl and PEG being added to wash and elution buffers improved separation of the target antibody monomer from aggregates. In comparison with the control run, monomer in the eluate was improved from 91.1% to 96.6%.

TABLE 2
Summary of monomer purity of elution fraction and elution pool
from five runs under different wash and elution conditions.

| | % Monomer | | | | |
|---|---|---|---|---|---|
| | | 5% | 5% | 3.5% | 600 |
| | 5% | PEG + 500 | PEG + 500 | PEG + 500 | mM |
| Fraction/pool | PEG | mM CaCl₂^{a} | mM NaCl^{a} | mM NaCl^{b} | NaCl^{a} |
| 1 | NA | 99.0 | 98.3 | 95.6 | 93.8 |
| 2 | NA | 95.4 | 97.5 | 95.9 | 90.5 |
| 3 | NA | 95.6 | 96.6 | 91.8 | 81.0 |
| 4 | NA | 95.9 | 97.0 | 90.1 | 84.6 |
| Pool | 80 | 96 | 97 | 93 | 88 |

^{a}Linear gradient elution.
^{b}Stepwise elution.

### CONCLUSION

In general Protein A chromatography does not provide good aggregate clearance under typical conditions. The present invention showed that PEG/calcium chloride and PEG/sodium chloride combination, when added to the mobile phase, significantly improves Protein A chromatography's aggregate removal capability. For the case used for method development and demonstration, the optimized procedure allows aggregates in Protein A elution pool to be reduced from 20% (control run) to approximately 3-4%.

In this case, the two different species need to be separated are monomer and aggregates, and the latter are known to bind tighter. In this study, the extent to which calcium chloride improves resolution between different species is less than that observed in a previous study. Nevertheless, the inventors learned that the calcium chloride mediated resolution-improving effect can be significantly enhanced by the presence of 5% PEG (FIG. 4B). It was further learned that PEG/sodium chloride combination can achieve similar effect (FIG. 6A), and sodium chloride by itself improves resolution to a larger extent than calcium chloride alone though the separation is also not complete.

The two salts (i.e., calcium chloride and sodium chloride) achieve resolution-enhancing effect through similar mechanisms. In either case, the salt affects monomer and aggregates to a different extent, resulting in improved resolution. PEG, although showed no effect on resolution by itself at up to 10%, can significantly improve chaotropic/kosmotropic salt mediated resolution-enhancing effect, allowing near-complete separation of monomer from aggregates.

In conclusion, the inventors developed a novel method that significantly improves Protein A chromatography's aggregate removing capability. This new method, by allowing the majority of aggregates to be removed at the Protein A capture step, significantly alleviates the burden on subsequent polishing steps and hence improves the overall robustness of downstream process.

1. A combination for use in Protein A chromatography comprising a component A which is at least one type of PEG polymer and a component B which is at least one Hofmeister series salt.

2. The combination of claim 1, consisting of a component A which is at least one type of PEG polymer and a component B which is at least one Hofmeister series salt.

3. The combination of claim 1, wherein the PEG polymer has a molecular weight of from about 200 Dalton to about 10,000,000 Dalton, preferably from about 400 Dalton to about 6000 Dalton.

4. The combination of claim 1, wherein the Hofmeister series salt is composed of a combination of Hofmeister series of cations and inions, preferably is one salt selected from the group consisting of calcium chloride, sodium chloride, magnesium chloride and potassium chloride.

5. A composition or a kit comprising the combination of claim 1.

6. The composition or the kit of claim 5, further comprising a component C, wherein the component C is one buffer selected from the group consisting of wash buffer solution and elution buffer solution.

7. The composition or the kit of claim 6, wherein the wash buffer solution or the elution buffer solution comprises NaAc and/or HAc.

8. The composition or the kit of claim 6, wherein ratio of weight of PEG polymer relative to the volume of the wash buffer solution or elution buffer solution is from about 10 g:1 L to about 100 g:1 L, preferably from about 20 g:1 L to about 50 g:1 L.

9. The composition or the kit of claim 6, wherein ratio of molar quantity of the Hofmeister series salt relative to the volume of the wash buffer solution or elution buffer solution is about 250 mmol:1 L and more, preferably is from about 250 mmol:1 L to about 1 mol:1 L, more preferably is from about 500 mmol:1 L to 750 mmol:1 L.

10. Use of the combination of claim 1 or the composition or the kit of claim 5 in protein purification by Protein A chromatography.

11. A method for improving aggregate removal by Protein A chromatography, comprising the following steps:
1) loading a protein sample onto a Protein A chromatography column,
2) washing the column with a wash buffer, wherein the wash buffer comprises at least one type of PEG polymer and at least one Hofmeister series salt, and
3) eluting the column with an elution buffer, wherein the elution buffer comprises at least one type of PEG polymer and at least one Hofmeister series salt.

12. The method of claim 11, wherein the PEG polymer has a molecular weight of from about 200 Dalton to about 10,000,000 Dalton, preferably from about 400 Dalton to about 6000 Dalton.

13. The method of claim 11, wherein the Hofmeister series salt is composed of a combination of Hofmeister series of cations and inions, preferably is one salt selected from the group consisting of calcium chloride, sodium chloride, or magnesium chloride, and potassium chloride.

14. The method of claim 11, wherein the wash buffer further comprises NaAc and HAc.

15. The method of claim 11, wherein the elution buffer further comprises HAc.

16. The method of claim 11, wherein percentage of weight of PEG polymer in the volume of the wash buffer solution or elution buffer solution is from about 1 w/v % to about 10 w/v %, preferably from about 2 w/v % to about 5 w/v %, depending on the molecular weight of the particular PEG being used.

17. The method of claim 11, wherein the percentage of molar quantity of the Hofmeister series salt in the volume of the wash buffer solution or elution buffer solution is about 250 mM and more, preferably is from about 250 mM to about 1 M, more preferably is from about 500 mM to 750 mM.
